(19) United States
(12) Patent Application Publication
(10) Pub. No.: US 2018/0059110 A1
(43) Pub. Date: Mar. 1, 2018

(54) METHOD AND SYSTEM FOR DETECTING AVIAN INFLUENZA VIRUS BASED ON CELL LINES

(71) Applicant: KOREA INSTITUTE OF SCIENCE AND TECHNOLOGY, Seoul (KR)

(72) Inventors: Jun-Seok LEE, Seoul (KR); Se-Young JANG, Seoul (KR); Seok LEE, Seoul (KR)

(21) Appl. No.: 15/479,124

(22) Filed: Apr. 4, 2017

(30) Foreign Application Priority Data
Aug. 31, 2016 (KR) 10-2016-0111580

(51) Int. Cl. G01N 33/569

(52) U.S. Cl. CPC G01N 33/56983; G01N 2333/11

(57) ABSTRACT

Provided is a method and a system for detecting avian influenza virus. More particularly, the method and a system for detecting avian influenza virus based on cell lines, which are capable of identifying avian influenza virus using infection pattern information for the cell lines, which is obtained by patterning a difference in infectivity of each subtype of avian influenza virus for some particular cells.

### CROSS REFERENCE TO RELATED APPLICATION

The present application claims the benefit of Korean Patent Application No. 10-2016-0111580 filed on Aug. 31, 2016.

### BACKGROUND OF THE INVENTION

### 1. Field of the Invention

The present invention relates to a method and a system for detecting avian influenza virus. More particularly, the present invention relates to a method and a system for detecting avian influenza virus based on cell lines, which are capable of identifying avian influenza viruses using infection pattern information for the cell lines, which is obtained by patterning differences in the infectivity of each subtype of avian influenza virus for some particular cells.

### 2. Description of the Related Art

Outbreaks of avian influenza (also known as bird flu) have been continuously reported. In this regard, the early detection of subtypes of avian influenza virus is critical to prevent further spread of the virus and minimize economical losses.

There are many different subtypes of avian influenza virus (AIV). AIV subtypes can be detected through embryo inoculation, which is a standard diagnostic method for avian influenza, performed by inoculating a sample taken from avian species into an embryonated egg and identifying an AIV subtype, as described in the following reference: Pereira H G, Lang G, Olesiuk O M, Snoeyenbos G H, Roberts D H, Easterday B C. New antigenic variants of avian influenza A viruses. Bull World Health Organ. 1966; 35(5):799-802.

However, the conventional embryo inoculation method is problematic in that it takes a long time, from 5 to 7 days, to inoculate a sample in ovo, incubate the embryonated egg and finally confirm a diagnose of avian influenza.

Thus, there is increasing need for a diagnostic technique capable of rapidly and accurately identifying subtypes of avian influenza virus prior to final characterization of AIV subtypes through embryo inoculation.

### SUMMARY OF THE INVENTION

Accordingly, the present invention has been made keeping in mind the above problems encountered in the related art, and the present invention is intended to provide a method and a system for detecting avian influenza virus based on cell lines, which enable the rapid and accurate identification of avian influenza virus.

In addition, the present invention is intended to provide a method and a system for detecting avian influenza virus based on cell lines, which are capable of detecting and identifying each subtype of avian influenza virus using infection pattern information for the cell lines, which is obtained by patterning the difference in infectivity of each subtype of avian influenza virus for some particular cells.

Therefore, the present invention provides a method and a system for detecting avian influenza virus based on cell lines, which include the following constructions.

In accordance with one embodiment of the present invention, the cell line-based method for detecting avian influenza virus is characterized by identifying the avian influenza virus using infection pattern information for cell lines, which is obtained by patterning differences in infectivity of each subtype of avian influenza virus for a particular cell.

In accordance with another embodiment of the present invention, the cell line-based method for detecting avian influenza virus is characterized by comprising the steps of reacting a sample with the particular cell in which the infectivity of each subtype of avian influenza virus is previously analyzed and of characterizing the virus by measuring an infection rate of the particular cell by the virus and comparing it with the cell line-infection pattern information to detect and identify each subtype of avian influenza virus.

In accordance with a further embodiment of the present invention, the cell line-based method for detecting avian influenza virus is characterized by further comprising a step of generating information to generate the cell line-infection pattern information, wherein the information-generating step includes the steps of selecting a susceptible cell having an infection rate with avian influenza virus above a predetermined level; and analyzing the information by analyzing the selected cell for infection rates by each subtype of AIV, quantifying the infection rates and patterning differences in infectivity of each subtype of AIV for the particular cell.

In accordance with yet another embodiment of the present invention, the cell line-based method for detecting an avian influenza virus is characterized in that the cells are cancer cells derived from any of a plurality of organs.

In accordance with still another embodiment of the present invention, the cell line-based system for detecting avian influenza virus (AIV) is characterized in that it comprises a cell line set, which includes a particular cell for which the infectability with each subtype of AIV is previously analyzed; and a detection device with which a viral infection rate of the particular cell is measured after a sample is reacted with the cell line set to characterize avian influenza virus.

In accordance with still another embodiment of the present invention, the cell line-based system for detecting AIV is characterized in that the detection device includes a measurement unit for calculating the viral infection rate for the particular cell; a storage unit for storing cell line-infection pattern information, which is obtained by patterning the difference in infectivity of each subtype of AIV for the particular cell; and a determination unit for comparing the infection rate calculated by the measurement unit with the cell line-infection pattern information so as to detect and identify each subtype of AIV.

The present invention can achieve the following effects by exemplary embodiments as well as constructions, combinations and usage relationships described below.

The present invention has the effect of enabling the rapid and accurate identification of an avian influenza virus.

The present invention also has the effect of enabling the detection and identification of each subtype of avian influenza virus using a cell line-infection pattern information, which is obtained by patterning the difference in infectivity of each subtype of AIV for some particular cells.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows confocal microscope images showing infection rates of MDCK cells with AIV subtypes; and

FIG. 2 is a table showing the infection pattern in which the infectivity of each subtype of AIV in particular cell lines is presented.

### DESCRIPTION OF SPECIFIC EMBODIMENTS

Hereinafter, a detailed description will be given of a method and a system for detecting avian influenza virus based on cell lines according to the present invention, with reference to the appended drawings. Unless otherwise defined, all terms used herein have the same meaning as commonly understood by one of ordinary skill in the art to which the present invention belongs. If the meaning of the term used herein conflicts with the general meaning thereof, it accords to the definition used herein. In the following description of the present invention, detailed descriptions of known constructions and functions incorporated herein will be omitted when they may make the gist of the present invention unclear. As used herein, when any part “comprises” or “includes” any element, it means that other elements are not precluded but may be further included, unless otherwise mentioned.

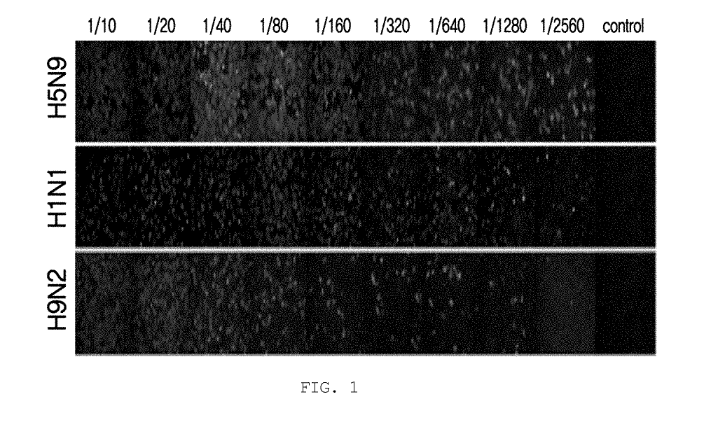

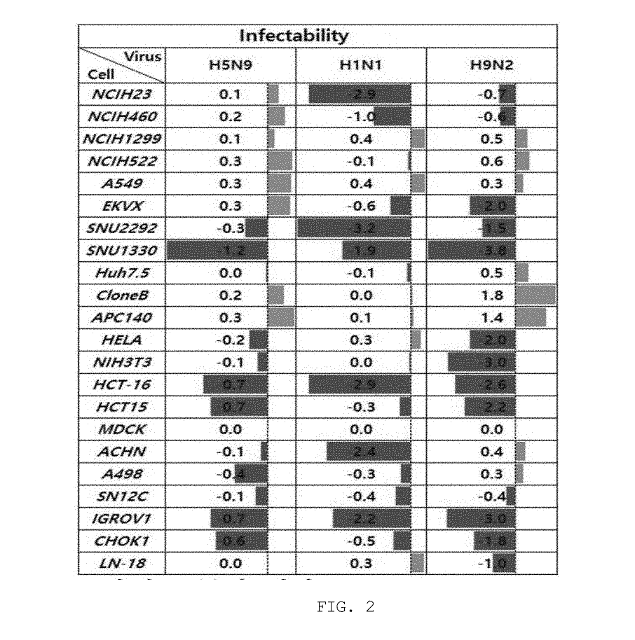

In one aspect, the present invention provides a method for detecting avian influenza virus (AIV) based on cell lines. Referring to FIGS. 1 and 2, the AIV detection method is based on the difference in infectability with each subtype of AIV for some particular cells. According to the present invention, the differential infectivity is patterned to generate cell line-infection pattern information, which enables the identification and sensing of AIV. A variety of cells is available, and for example, cancer cells derived from a plurality of organs may be used.

As described above, it is very difficult to detect and distinguish multiple subtypes of AIV. In this regard, the present invention employs a cell line-infection pattern information, which is obtained by patterning the difference in infectivity of each subtype of AIV for some particular cells. According to the present invention, AIV subtypes may be identified (or distinguished) by reacting a sample with a particular cell in which the infectivity of each subtype of AIV is previously analyzed and measuring a viral infection rate of the particular cell and comparing it with the cell line-infection pattern information. There are many different H and N subtypes of avian influenza A viruses, and their combinations result in multiple different subtypes of AIV. Such AIV subtypes display different infectivity in some particular cells. This varying host specificity of AIV is thought to come from the difference in the binding preference for sialic acid of glycans on the cell surface.

That is, the technical spirit of the present invention is to detect AIV infection in a variety of host cell lines having different glycan structures, select cells susceptible to AIV infection (having an infection rate above a predetermined level), pattern the difference in infectability with each subtype of AIV for some particular cells, and employ the thus generated cell line-infection pattern information. Based on this principle, a biological specimen may be analyzed to selectively identify a particular subtype of AIV.

Based on the above-described technical spirit, the method for detecting avian influenza virus will be described in detail as follows. The AIV detection method comprises the steps of generating information by patterning the difference in infectivity of each subtype of AIV for some particular cells to generate cell line-infection pattern information; reacting a sample with a particular cell in which the infectivity of each subtype of AIV is previously analyzed; and characterizing a virus by measuring an infection rate of the particular cell by the virus and comparing it with the cell line-infection pattern information to detect and identify each subtype of AIV.

The information-generating step, at which the difference in infectivity of each subtype of AIV for some particular cells is patterned to generate cell line-infection pattern information, may include the steps of selecting cells susceptible to AIV infection and analyzing the information.

At the step of selecting susceptible cells, cells having an infection rate with avian influenza virus above a predetermined level are selected. Since not all kinds of cells are susceptible to avian influenza virus, a wide variety of cells is subjected to infection with avian influenza virus, and susceptible cells, which have an infection rate above a predetermined level, are selected.

At the information-analyzing step, the selected cells are analyzed for infection rates with each subtype of AIV, and the infection rates are quantified to pattern the difference in infectivity of each subtype of AIV for the particular cells, thereby generating cell line-infection pattern information. For example, if some particular cells (e.g., NCIH1299, Clone-B and APC140) are susceptible to infection by AIV subtypes (e.g., H5N9, H1N1 and H9N2), cell line-infection pattern information, at the information-analyzing step, is generated, for instance, as described in Table 1, below (under the assumption that a1, a2, a3, b1, b2, b3, c1, c2 and c3 represent infectivity values).

TABLE 1

| | Infection rates | | |
|---|---|---|---|
| Cell line | H5N9 | H1N1 | H9N2 |
| NCIH1299 | a1 | a2 | a3 |
| CloneB | b1 | b2 | b3 |
| APC140 | c1 | c2 | c3 |

At the sample reaction step, a sample is made to react with a particular cell in which infectability with each subtype of AIV has been previously analyzed and of which infection patterns are present in the cell line-infection pattern information.

At the virus-characterizing step after the sample reaction step, the infection rate of the particular cell with the virus is measured and compared with the cell line-infection pattern information to detect and identify each subtype of AIV. For example, when NCIH1299 cells are used as the particular cell to be reacted with a sample and display an infection rate of a2 value, the sample is determined to contain AIV HIN1 subtype. At this step, the infectivity of AIV for cells may be measured by any of various conventional methods.

In another aspect, the present invention provides a system for detecting avian influenza virus based on cell lines. The AIV detection system may include a cell line set (not shown) and a detection device (not shown).

The cell line set includes some particular cells (cell lines) in which the infectivity of each subtype of AIV is previously analyzed. The cell line set may be composed of a single cell line or multiple different cell lines. When the cell line set is composed of multiple different cell lines, the infectivity of the virus is measured in each cell line and compared with a cell line-infection pattern information, thereby more accurately subtyping avian influenza virus.

The detection device serves to characterize AIV by measuring a viral infection rate of the particular cell after a sample is reacted with the cell line set. The detection device includes a measurement unit for calculating the viral infection rate for the particular cell; a storage unit for storing cell line-infection pattern information, which is obtained by patterning the difference in infectivity of each subtype of AIV for the particular cell; and a determination unit for comparing the infection rates calculated by the measurement unit with the cell line-infection pattern information so as to detect and identify each subtype of AIV.

A better understanding of the present invention may be obtained through the following examples which are set forth to illustrate, but are not to be construed as the limit of the present invention.

### Example 1: Cell Culture

(1) A variety of cell lines were used as follows: eight lung cancer cell lines (NCI-H23, NCI-H522, NCI-H460, NCI-H1299, EKVX, SNU-2292, SNU-1330 and A549), two colon cancer cell lines (HCT16 and HCT15), two ovarian cancer cell line (IGROV1, CHO-K1), a human cervical cancer cell line (HeLa), three hepatic carcinoma cell lines (Huh7.5, APC140 and Clone-B), four kidney-derived cell lines (A498, SN12C, ACHN and MDCK), a malignant glioma cell line (LN-18), and an embryonic fibroblast cell line (NIH-3T3).

(2) Each cell line was seeded in a 96-well plate and cultured in Dulbecco's Modified Eagle Medium (DMEM) at 37° C. for 24 hrs.

### Example 2: Evaluation for Infectability with AIV of Various Cell Lines

(1) The cultured cell lines were infected with three two-fold-diluted subtypes of AIV (H5N9, H1N1 and H9N2) for 24 hrs (before dilution, the virus titer was 10^{7.625} EID₅₀/ml), and then analyzed for infection rates with each subtype of AIV. The infectability with AIV subtypes of each cell line was measured by immunofluorescence analysis using a mouse primary antibody recognizing the influenza virus nucleoprotein (NP) and a fluorescence-conjugated secondary antibody specific to the primary antibody. In detail, after the cells were infected with the virus, the medium containing the virus was removed, and cells were fixed in 100% methanol for 5 min and permeabilized with 0.25% Triton X-100 at room temperature for 10 min so as to increase the cell permeability of antibodies. The fixed cells were then blocked with 2.5% fetal bovine serum (FBS) for 2 hrs to block non-specific antibody binding. Sequentially, cells were immunostained against the avian influenza viral nucleoprotein (NP). Cells were incubated with an anti-NP primary antibody (diluted 1:1000), followed by the addition of a secondary antibody conjugated with Alexa Fluor 488 (diluted 1:2500). Cell nuclei were counterstained with fluorescent Hoechst dye. Then, fluorescence images were taken using a confocal microscope, and the infection rates of cells with the virus were calculated by analyzing the images. FIG. 1 shows confocal microscope images of MDCK cells (avian influenza virus was diluted starting from 1/10 to 1/2560). The infectivity of the influenza A viruses in cells was determined based on the percentage of infected cells. The infectivity (%) was calculated using a cell profiler software, with which nuclei and a viral protein of infected cells are designated using a primary object module and the infectivity was then calculated through a measurement module according to Equation 1, below. After the infectivity of AIV in particular cell lines was obtained using Equation 1, values are shown on a log 2 scale and are represented relative to that in MDCK cells. The relative infectivity values are presented in FIG. 2. For MDCK cells, serving as a standard control, a value of 0 was set as a reference score, such that values above zero would be scored “+” and values below zero would be scored “−”. Blue (positive) indicates higher infectivity of AIV and red (negative) indicates lower infectivity, relative to that of MDCK cells.

Infectivity (%)=(the number of overlapped nuclei with viral proteins/the total number of nuclei)×100 [Equation 1]

(2) As seen in FIG. 2, some cell lines were susceptible to infection with avian influenza viruses while other cell lines were not susceptible. Also, there was a remarkable difference in infection rate among susceptible cell lines. Based on these results, in the present invention, cell line-infection pattern information is generated, a sample is reacted with particular cells of which infection patterns are saved in the information, and calculated infection rates of the cells are compared with the cell line-infection pattern information, thereby identifying a specific subtype of avian influenza virus.

Although a variety of embodiments of the present invention have been disclosed for illustrative purposes, those skilled in the art will appreciate that various modifications, additions and substitutions are possible, without departing from the scope and spirit of the invention as disclosed in the accompanying claims.

1-4. (canceled)

5. A system for detecting avian influenza virus (AIV) based on a cell line, comprising:
a cell line set, which includes a particular cell in which infectivity of each subtype of AIV is previously analyzed; and
a detection device with which a viral infection rate of the particular cell is measured after a sample is reacted with the cell line set to characterize avian influenza virus,
wherein the detection device comprises:
a measurement unit for calculating the viral infection rate for the particular cell;
a storage unit for storing a cell line-infection pattern information, which is obtained by patterning a difference in infectivity of each subtype of AIV for the particular cell; and
a determination unit for comparing the infection rate calculated by the measurement unit with the cell line-infection pattern information to detect and identify each subtype of AIV.

6. (canceled)

7. The system of claim 5, wherein the cell is a cancer cell derived from any of a plurality of organs.
